(19) United States
(12) Patent Application Publication
(10) Pub. No.: US 2006/0100449 A1
(43) Pub. Date: May 11, 2006

(54) Integrated two-step process for the production of gamma-methyl-alpha-methylene-gamma-butyrolactone from levulinic acid and hydrogen

(76) Inventor: Leo Ernest Manzer, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/270,038

(22) Filed: Nov. 9, 2005

Related U.S. Application Data
(60) Provisional application No. 60/626,583, filed on Nov. 10, 2004.

(51) Int. Cl. C07D 305/12

(52) U.S. Cl. 549/326

(57) ABSTRACT

An integrated two step method for making gamma-methyl alpha-methylene-gamma-butyrolactone (meMBL) from levulinic acid, in which levulinic acid is reacted with hydrogen in the first step to form a crude reaction product containing gamma-valerolactone, and the crude reaction product, without removal of unreacted levulinic acid therefrom, is reacted with formaldehyde in the second step to produce MeMBL.

### CROSS-REFERENCE TO RELATED APPLICATION

This application claims priority under 35 U.S.C. §119 from U.S. Provisional Application Ser. No. 60/626,583, filed Nov. 10, 2004.

### FIELD OF THE INVENTION

The present invention relates to a method for making gamma-methyl-alpha-methylene-gamma-butyrolactone (MeMBL) using an integrated two step process, the first step of which involves a reaction in which levulinic acid (LA) is reacted with hydrogen in the presence of a catalyst to yield a crude, two phase reaction product, the liquid phase of which contains gamma-valerolactone (GVL), and the second step of which involves contacting the liquid phase, without removal of any unreacted LA therefrom, with formaldehyde in the presence of a catalyst to yield a reaction product that contains MeMBL.

### BACKGROUND

It is known that levulinic acid can be reacted with hydrogen in the presence of a suitable catalyst to produce a product that contains gamma-valerolactone (hereinafter “GVL”). It is also known that GVL can be reacted with formaldehyde in the presence of a suitable catalyst to produce a reaction product that contains gamma-methyl-alpha-methylene-gamma-butyrolactone (hereinafter “MeMBL”). Generally, the product of the levulinic acid conversion will be subjected to various purification procedures to produce a purified GVL, and the purified GVL will, in turn, be used as a reactant to produce MeMBL. It might be expected that the use of crude, i.e., unpurified GVL as a reactant to make MeMBL would result in compromised yields of MeMBL, possibly because of the presence of trace impurities that could deactivate or otherwise adversely affect the catalyst used to convert the GVL into MeMBL.

### SUMMARY OF THE INVENTION

Surprisingly, it has now been found that the crude GVL-containing reaction product of the levulinic acid conversion reaction can be used directly, without removal of unreacted levulinic acid therefrom, as a reactant in the GVL to MeMBL conversion reaction, without compromising the conversion and selectivity of the GVL reaction, when compared to a reaction in which purified GVL was utilized.

More specifically, the present invention is a method for making gama-methyl-alpha-methylene-gama-butyrolactone (MeMBL), comprising:

(a) contacting in a first reactor levulinic acid and hydrogen, optionally in a solvent, at a temperature between 100 and 300 degrees C. and a pressure between 50 and 3000 psi (0.4 and 21 MPa) in the presence of a catalyst capable of converting the levulinic acid and hydrogen into gamma-valerolactone (GVL), for a period of time sufficient to achieve at least 95% conversion of levulinic acid, to form a first reaction product that comprises GVL and unreacted hydrogen, and any unreacted levulinic acid;

(b) separating the reaction product into a gas phase comprising a major portion of the unreacted hydrogen, and a liquid phase comprising a major portion of the GVL;

(c) introducing into a second reactor (i) the liquid phase, without any removal of levulinic acid therefrom, and (ii) a formaldehyde source capable of forming formaldehyde, said second reactor containing a catalyst comprising a silica support and at least one element selected from the group consisting of Rb, K, Cs and Ba, to form a second reaction product comprising MeMBL; and

(d) optionally separating the MeMBL from the second reaction product.

### BRIEF DESCRIPTION OF THE DRAWING

The Drawing consists of two figures.

FIG. 1 depicts in schematic form a preferred embodiment of the present invention.

FIG. 2 is a graph showing the results of the process in accordance with the present invention, compared to data obtained from a two step process in which the GVL-containing product of the first step is purified prior to reaction in the second step.

### DETAILED DESCRIPTION OF THE INVENTION

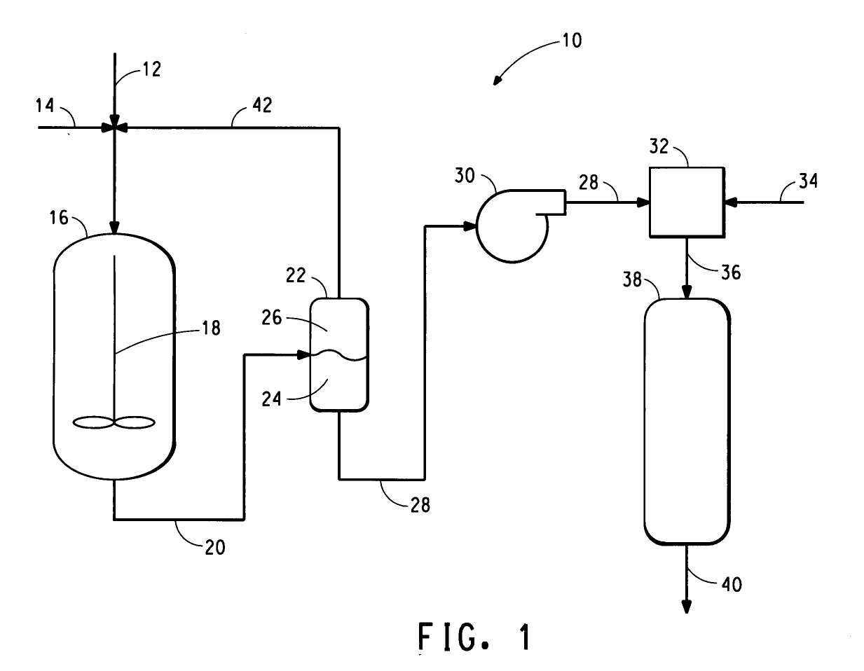

Referring now to FIG. 1, there is shown in schematic form apparatus 10 for practicing the method of the present invention.

A stream 12 comprising levulinic acid and a stream 14 of gaseous hydrogen are fed to first reactor 16 containing optional mixing device 18 and a suitable catalyst (not shown). Suitable reactors include trickle bed, autoclave (batch or continuous stirred tank), and fixed bed reactors. The reactor should be configured to provide for adequate mixing of the hydrogen, levulinic acid and catalyst. One way to achieve this is to employ a mechanical agitator in a suitable autoclave.

Suitable catalysts include one or more elements selected from the group consisting of palladium, ruthenium, rhenium, rhodium, iridium, platinum, nickel, cobalt, copper, iron, and osmium. The catalytic element can optionally be supported on a support. The support can be in the form of powder, granules, pellets, or the like. The supported catalyst can be made by depositing either the element itself, or a compound thereof, on a suitable support material. The depositing can be accomplished by a number of well-known methods. A preferred support material can be selected from the group consisting of carbon, alumina, silica, silica-alumina, silica-titania, titania, titania-alumina, barium sulfate, calcium carbonate, strontium carbonate, and various zeolites. Most preferred supports are alumina, titania and carbon. The catalysts of the present invention may optionally comprise catalyst additives and promoters that will enhance the efficiency of the catalyst. In the processes of the invention, the preferred catalytic element content range of the supported catalyst is from about 0.1% to about 20% of the supported catalyst based on element weight plus the support weight. A more preferred catalytic element content range is from about 1% to about 10% of the supported catalyst. A further preferred catalytic metal content range is from about 1% to about 5% of the supported catalyst.

Other suitable unsupported catalysts include the so-called Raney® catalysts. Raney® catalysts have a high surface area due to selectively leaching an alloy containing the active element(s) and a leachable elementl (usually aluminum). Raney® catalysts have high activity due to the higher specific area and allow the use of lower temperatures in hydrogenation reactions. The active metals of Raney® catalysts include nickel, copper, cobalt, iron, rhodium, ruthenium, rhenium, osmium, iridium, platinum, palladium, compounds thereof and combinations thereof. Promoter metals may also be added to the base Raney® metals (available from W.R. Grace & Co., Columbia Md.) listed above to affect selectivity and/or activity of the Raney® catalyst. Promoter metals for Raney® catalysts may be selected from transition metals from Groups 3 through Group 8, and Group 11 and Group 12 of the Periodic Table of the Elements. Examples of promoter metals for the Raney® based catalytic metal include chromium, molybdenum, platinum, rhodium, ruthenium, osmium, and palladium, typically at about 2% by weight of the total metal.

The temperature and pressure conditions of reactor 16 should be in the range of 100° C. and 300° C. and between 50 and 3000 psi (0.4 and 21 MPa). Either or both of the levulinic acid 12 and hydrogen 14 can be preheated before being introduced into reactor 16. Alternatively, they can be introduced into reactor 16 and heated and pressurized in situ. The temperature, pressure, catalyst loading and hold-up time should be selected to achieve at least 95% conversion of levulinic acid to GVL. One can determine the appropriate combination of conditions to achieve this level of conversion by running independent experiments in the reactor 16 that one chooses to employ in the method of the present invention.

The reaction product of reactor 16 (without the catalyst) is withdrawn as a stream 20 that is then introduced into a separator 22. The separator 22 typically is a non-agitated vessel or tank that allows the reaction product to separate into two phases, (i) a liquid phase 24 that contains the major portion of the GVL produced in reactor 16, and any unreacted levulinic acid, and (ii) a vapor phase 26 that contains the major portion of unreacted hydrogen, which can optionally be recycled back to reactor 16 as stream 42.

A steam 28 of the liquid phase 24 is withdrawn from separator 22. The stream 28—without any removal of levulinic acid therefrom—is pumped using pump 30 into a mixer 32 along with a stream 34 of a “formaldehyde source.” A “formaldehyde source” is a material that is capable of forming formaldehyde under the conditions present for the second phase of the present method, i.e., the conversion of GVL into MeMBL. Suitable formaldehyde sources include, but are not limited to aqueous formalin, a hemiacetal of an alcohol, a low molecular weight polyformaldehyde, or formaldehyde trimer (trioxane). Formalin is preferred because it is the lowest cost source of formaldehyde. The use of trimers and oligomers reduces the need to remove water from the process. Anhydrous formaldehyde can also be used under appropriate conditions to minimize polymerization. Hemiacetals work effectively, but require separate steps to release formaldehyde from the alcohol and to recover and recycle the alcohol.

The mixture 36 of formaldehyde source 34 and stream 28 is then introduced into second reactor 38. Suitable reactors include, but are not limited to, fixed bed and fluidized bed reactors. Reactor 38 must contain a catalyst (not shown) that is capable of catalyzing the conversion of GVL to MeMBL. Suitable catalysts comprise a silica support and at least one element selected from the group consisting of potassium, rubidium, cesium and barium. The silica support optionally may be doped with aluminum, zirconium and/or titanium. These catalysts preferably contain from 0.1 to 40 wt % of the catalytic element relative to the combined weight of the support plus the element (as opposed to the compound of which the element is a part). Preferably the support is porous and has a pore size distribution such that pores having a diameter between 65 and 3200 Angstroms contribute a pore volume of at least 0.3 cubic centimeters per gram of catalyst. This requirement can be ascertained by using mercury or nitrogen porosimetry.

The temperature and pressure of reactor 38 should be chosen to maximize the conversion of GVL into MeMBL. Typical temperatures range from 250° C. to 400° C. Typical pressures range from 15 to 100 psi (0.2 to 0.8 MPa). One can determine the appropriate combination of conditions, including catalyst contact time, to achieve high yields of MeMBL by running independent experiments in whatever reactor 38 one chooses to employ in the method of the present invention.

The product of the reaction in reactor 38 is withdrawn from the reactor as stream 40, which can be subject to methods known in the art for separating MeMBL from unreacted GVL and formaldehyde source. A particularly suitable method for separating MeMBL from unreacted GVL involves polymerizing the MeMBL in the GVL solution using standard free radical polymerization, followed by precipitation of the poly-MeMBL, followed by thermal decomposition of the poly-MeMBL back to monomeric MeMBL. Another effective method is liquid/liquid extraction.

### EXAMPLES

### Example

### Present Process Without Purification of GVL

A 1-litre stainless steel autoclave, equipped with a glass liner, was charged with 510.4 grams of levulinic acid and 2.98 grams of 5% Ru/Al₂O₃ (AP38 from Engelhard Corp). The autoclave was pressurized to 500 psig (3.5 MPa) with hydrogen gas and heated to 200° C. for 2 hours. The pressure was maintained at 3.34 MPa during the course of the experiment. At the end of 2 hours, the reactor was cooled and vented. Analysis of the product showed 100% conversion of levulinic acid with greater than 98% selectivity to gamma-valerolactone (GVL).

The product was filtered from the catalyst, and without further purification, the GVL was added to formalin (37% aqueous formaldehyde) to prepare a solution with a molar ratio of formaldehyde to GVL of 4:1. This solution was fed to a vaporizer (held at 200° C.) followed by the introduction of nitrogen, to carry the vapor through a ¼ inch tubular reactor containing 2 cubic centimeters of catalyst composition comprising 20% Rb(acetate) supported on Engelhard KA-160 SiO₂ (Engelhard L-6803-07A), particle size of 20-30 mesh, heated to 325° C. The nitrogen flow rate was 24 cubic centimeters/minute, and the liquid feed rate was 1.12 ml/hour. The reactor effluent was condensed in a cold trap and analyzed off-line by gas chromatography using an internal standard. Conversion was based on the weight percent of GVL converted. Selectivity was based on the weight fraction of MeMBL (and its isomers) relative to the amount of GVL converted. Results are shown in the table below, in which “TOS” means time on stream.

| | TOS | % GVL | % MeMBL |
|---|---|---|---|
| Catalyst | (hours) | Conversion | Selectivity |
| 20% Rb/KA-160 20-30 | 0.08 | 59.00 | 97.85 |
| mesh(L-6803-07A) | | | |
| 20% Rb/KA-160 20-30 | 0.25 | 53.51 | 94.37 |
| mesh(L-6803-07A) | | | |
| 20% Rb/KA-160 20-30 | 0.50 | 41.98 | 94.33 |
| mesh(L-6803-07A) | | | |
| 20% Rb/KA-160 20-30 | 0.75 | 35.75 | 94.49 |
| mesh(L-6803-07A) | | | |
| 20% Rb/KA-160 20-30 | 1.00 | 34.06 | 93.43 |
| mesh(L-6803-07A) | | | |
| 20% Rb/KA-160 20-30 | 1.25 | 30.95 | 94.59 |
| mesh(L-6803-07A) | | | |
| 20% Rb/KA-160 20-30 | 1.50 | 28.42 | 95.00 |
| mesh(L-6803-07A) | | | |
| 20% Rb/KA-160 20-30 | 2.00 | 25.82 | 94.14 |
| mesh(L-6803-07A) | | | |
| 20% Rb/KA-160 20-30 | 2.50 | 23.43 | 95.43 |
| mesh(L-6803-07A) | | | |
| 20% Rb/KA-160 20-30 | 3.00 | 21.82 | 95.61 |
| mesh(L-6803-07A) | | | |
| 20% Rb/KA-160 20-30 | 4.25 | 18.62 | 95.72 |
| mesh(L-6803-07A) | | | |
| 20% Rb/KA-160 20-30 | 5.00 | 18.77 | 95.91 |
| mesh(L-6803-07A) | | | |

### Comparative Example

### Two Step Process with Intermediate Purification of GVL

A 1-litre stainless steel autoclave, equipped with a glass liner, was charged with 496.3 grams of levulinic acid and 2.98 grams of 5% Ru/Al₂O₃ (AP38 from Engelhard Corp). The autoclave was pressurized to 500 psig (3.5 MPa) with hydrogen gas and heated to 200° C. for 2 hours. The pressure was maintained at 3.34 MPa during the course of the experiment. At the end of 2 hours, the reactor was cooled and vented. Analysis of the product showed 100% conversion of levulinic acid, with greater than 98% selectivity to gamma-valerolactone (GVL).

The product from the hydrogenation reaction was filtered from the catalyst, and the GVL was purified by distillation. The distilled GVL was added to formalin (37% aqueous formaldehyde) to prepare a solution with a molar ratio of formaldehyde to GVL of 4:1. This solution was fed to a vaporizer (held at 200° C.) followed by the introduction of nitrogen to carry the vapor through a ¼ inch tubular reactor containing 2 cubic centimeters of catalyst composition comprising 20% Rb(acetate)/supported on Engelhard KA-160 SiO₂ (L-6803-07A), particle size of 20-30 mesh, heated to 325° C. The nitrogen flow rate was 24 cubic centimeters/minute, and the liquid feed rate was 1.12 ml/hour. The reactor effluent was condensed in a cold trap and analyzed off-line by gas chromatography using an internal standard. Conversion was based on the weight percent of GVL converted. Selectivity was based on the weight fraction of MeMBL (and its isomers) relative to the amount of GVL converted. Results are shown in the table below, in which “TOS” means time on stream.

| | TOS | % GVL | % MeMBL |
|---|---|---|---|
| Catalyst | (hours) | Conversion | Selectivity |
| 20% Rb/KA-160 20-30 | 0.25 | 42.01 | 95.97 |
| mesh(L-6803-07A) | | | |
| 20% Rb/KA-160 20-30 | 0.5 | 36.94 | 94.84 |
| mesh(L-6803-07A) | | | |
| 20% Rb/KA-160 20-30 | 0.75 | 31.69 | 94.44 |
| mesh(L-6803-07A) | | | |
| 20% Rb/KA-160 20-30 | 1.08 | 28.05 | 94.45 |
| mesh(L-6803-07A) | | | |
| 20% Rb/KA-160 20-30 | 1.25 | 26.53 | 94.28 |
| mesh(L-6803-07A) | | | |
| 20% Rb/KA-160 20-30 | 1.5 | 25.19 | 94.72 |
| mesh(L-6803-07A) | | | |
| 20% Rb/KA-160 20-30 | 2 | 23.33 | 94.87 |
| mesh(L-6803-07A) | | | |
| 20% Rb/KA-160 20-30 | 2.58 | 22.10 | 95.00 |
| mesh(L-6803-07A) | | | |
| 20% Rb/KA-160 20-30 | 3.83 | 20.42 | 95.19 |
| mesh(L-6803-07A) | | | |
| 20% Rb/KA-160 20-30 | 4.5 | 20.23 | 95.19 |
| mesh(L-6803-07A) | | | |
| 20% Rb/KA-160 20-30 | 5 | 19.77 | 95.26 |
| mesh(L-6803-07A) | | | |

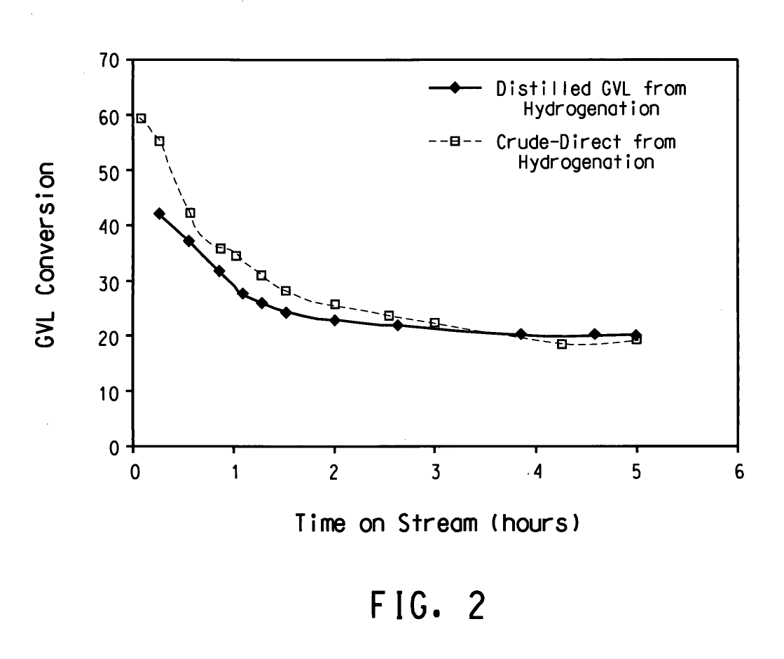

The data in the tables above are illustrated in FIG. 2, which shows that the method of the present invention, in which the unpurified product of the first reaction (conversion of levulinic acid to GVL) provides about the same (or possibly slightly higher, at least for the first three hours) conversion of GVL to MeMBL with time on stream in the second reaction, compared with that of the comparative example, in which the first reaction product was purified before reaction with formaldehyde in the second reaction.

1. A method for making gama-methyl-alpha-methylene-gama-butyrolactone (MeMBL), comprising:
(a) contacting in a first reactor levulinic acid and hydrogen, at a temperature between 100° C. and 300° C. and a pressure between 50 and 3000 psi (0.4 MPa and 21 MPa) in the presence of a catalyst capable of converting the levulinic acid and hydrogen into gamma-valerolactone (GVL), for a period of time sufficient to achieve at least 95% conversion of levulinic acid, to form a first reaction product that comprises GVL and unreacted hydrogen;
(b) separating the reaction product into a gas phase comprising a major portion of the unreacted hydrogen, and a liquid phase comprising a major portion of the GVL;
(c) introducing into a second reactor (i) the liquid phase, without any removal of, and (ii) a formaldehyde source capable of forming formaldehyde, said second reactor containing a catalyst comprising a silica support and at least one element selected from the group consisting of Rb, K, Cs and Ba, to form a second reaction product comprising MeMBL; and
(d) optionally separating the MeMBL from the second reaction product.

2. The method of claim 1 in which the catalyst in step (c) comprises Rb on a silica support.

3. The method of claim 1 in which the catalyst in step (c) comprises Ba on a silica support.

4. The method of claim 1 in which the catalyst in step (a) comprises Ru on a carbon or alumina support, and the catalyst of step (c) comprises K, Ba or Rb on a silica support.
